(19) United States
(12) Patent Application Publication
(10) Pub. No.: US 2026/0256766 A1
(43) Pub. Date: Sep. 3, 2026

(54) Nicotine Products

(71) Applicant: NOAT SWEDEN AB, Hjärnarp (SE)

(72) Inventors: Alexander HÄGGQVIST, Lausanne (CH); Erik HÖGFELDT, Hjärnarp (SE); Marie MANNERLÖF, Bjärred (SE)

(73) Assignee: NOAT SWEDEN AB, Hjärnarp (SE)

(21) Appl. No.: 19/489,227

(22) PCT Filed: May 31, 2024
(86) PCT No.: PCT/EP2024/065044

§ 371 Date: Dec. 1, 2025

(30) Foreign Application Priority Data
Jun. 2, 2023 (SE) 2330260-7

(51) Int. Cl. A61K 31/465; A61K 9/00; A61K 47/02; A61K 47/10; A61K 47/26; A61K 47/38

(52) U.S. Cl. CPC A61K 31/465; A61K 9/006; A61K 9/009; A61K 47/02; A61K 47/10; A61K 47/26; A61K 47/38

(57) ABSTRACT

The invention relates to an oral nicotine composition having improved characteristics as well as a pouch comprising said oral nicotine composition, wherein said oral nicotine composition comprises nicotine and oat fibers and/or oat powders.

### FIELD OF INVENTION

The invention relates to an oral nicotine composition having improved properties as well as a pouch comprising said oral nicotine composition, wherein said oral nicotine composition comprises nicotine and at least one filler.

### BACKGROUND OF INVENTION

Delivery of nicotine by smoking has many well-known drawbacks, in particular health related problems, including cancer.

Tobacco substitutes also suffer from disadvantages, such as inadequate relief of cravings for the recovering smoker as well as some health-related issues. EP3970513 disclose a nicotine composition free from tobacco, wherein the pouch do not contain the specific amount of filler and natural fiber, being important features to achieve the invented pouch and remove the problems with known pouches.

US2021307375 discloses a number of nicotine pouch compositions, wherein the pouch do not contain the specific amount of filler and natural fiber as disclosed herein which give a healthier product which gives a release of nicotine that relief to the consumer and still maintain its humidity to be a pleasant experience.

It is an object of the present invention to provide nicotine substitute in the form of an oral nicotine composition presented in a pouch that could eliminate or reduce the problems and unpleasant properties with the above, mentioned products.

### SUMMARY OF THE INVENTION

It is an objective of the present invention to mitigate, alleviate or eliminate one or more of the identified deficiencies in the art and disadvantages singly or in any combination and solve at least the mentioned problems.

In a first aspect the invention relates to an oral nicotine composition comprising, nicotine from 0.5 to 12 mg, and a filler in an amount of 30-75% by weight selected from the group consisting of microcrystalline cellulose, cellulose and cellulose derivatives, and mixtures thereof and a natural fiber in an amount of 5-15% by weight and substantially free from tobacco. The oral nicotine composition is preferably free from pH adjusting agent.

By such an oral nicotine composition a number of problems are solved, and a product provided having an improved sensory feeling to the consumer of said product. The oral nicotine composition provides a balanced/controlled release of nicotine and a prolonged craving relief.

The product gives a smooth and soft feeling upon being placed under the lip, which is obtained by the unique combination of the components present in the composition. This also makes the product suitable to be molded and added into a compartment, such as a pouch. In addition, the oral nicotine composition is improved compared to other comparable oral nicotine compositions on the market, the improvement is that the oral nicotine composition is less harmful to the mucous membranes and teeth in the mouth and provides a freshness, i.e., is free from irritating and/or corrosive excipients, such as pH adjusting agents, ammonium chloride and preservatives.

Further, another advantage is that the product has a prolonged taste as well as improved the storage capacity by keeping the humidity to an acceptable level during the storage which eliminate the problem with drying of the filler(s) and thus inhibit the feeling of a dry and sticky product. The feeling of the product is highly important for acceptance of the product by the consumer. Further by providing a perfect humidity as well as the presence of nicotine microbial growth is inhibited which increases the long-term storage.

In addition, all components of the oral nicotine composition have been chosen to provide a composition that uses less or no plastics and gives a lesser footprint to the environment, i.e., being environmentally friendly.

In another aspect the invention relates to a container, such as a pouch comprising said oral nicotine composition to be delivered to the oral cavity comprising, nicotine from 0.5 mg to 12 mg and a filler in an amount of 30-75% by weight selected from the group consisting of microcrystalline cellulose, cellulose and cellulose derivatives, and mixtures thereof and a natural fiber in an amount of 5-15% by weight and substantially free from tobacco.

By providing the oral nicotine container, such as a pouch, a product is provided that modulates release of water-soluble components from the composition situated within the cavity of the container such as a water-permeable pouch. Such release can be modulated, e.g., by modifying a packing density/molding of the composition and/or by modifying a shape of the outer water-permeable pouch or the material of the pouch.

### DETAILED DESCRIPTION OF THE INVENTION

### Definitions

As used herein, the definition “weight percent”, “% by weight, based on dry weight” and the like is intended to refer to the weight percent of the ingredient referred to on the basis of the total weight of the dry ingredients, i.e. all ingredients of the preparation, composition or product referred to excluding the moisture content. Thus, “% by weight, based on total weight” as used herein is the same as “% by weight, based on wet weight”.

As used herein the term “oral nicotine composition” refers to the composition suitable for use in an oral nicotine pouch, i.e., in pouches for oral use comprising nicotine. The oral nicotine composition can also be placed in other types of containers that can be placed in the oral cavity and sucked upon to release the nicotine present within the container, as long as the container is non-toxic, water-soluble and degradable.

As used herein the term “pouch” is intended to mean a container typically formed by a web of a fibrous material enclosing a cavity. The pouch is designed for administration of an active ingredient in the oral cavity, and thus it is adapted for oral use, it is non-toxic and might be biodegradable. The fibrous material may e.g., form a woven or non-woven web or fabric. The pouch may for example be sealed by bonding two corresponding pieces of web or fabric to each other along their edges to form a cavity for the nicotine and the water-soluble composition. In order to release the nicotine, the pouch is water-permeable to allow saliva from the oral cavity to penetrate the pouch and enter the cavity, where the saliva come in contact with the nicotine, whereby the nicotine is released from the oral pouch.

As used herein the term “pH adjusting agent or pH adjusting agents” is intended to mean one or more substances added with the purpose to adjust and control the pH of an aqueous liquid, such as saliva, when the product containing the pH adjusting agent is dissolved or dispersed in said aqueous liquid.

As used herein the term “nicotine” refers to any form of nicotine that includes both free-base nicotine extracted from tobacco as well as synthetically manufactured free-base nicotine, the free-base nicotine is not provided in the form of tobacco or powdered tobacco. Nicotine can also be chemically bound to a resin such as nicotinepolacrilex. Nicotine can also be a mixture of different nicotine forms.

As used herein the term “free-base nicotine” refers to non-protonated form of nicotine, and therefore does not include nicotine salts and nicotine provided as a complex between nicotine and an ion exchange resin. While free-base nicotine includes both free-base nicotine extracted from tobacco as well as synthetically manufactured free-base nicotine, the free-base nicotine is not provided in the form of tobacco or powdered tobacco. Typically, free-base nicotine is provided as a liquid.

As used herein the term “filler” is intended to mean a material that increase the volume of the composition. In one embodiment the filler is present to increase the volume of the container, such as pouches to be used under the lip.

As used herein the term “Substantially free from tobacco fibers” is intended to mean that less than 95% by weight of tobacco fibers or even less such as 96, 97, 98, 99% by weight or even free from tobacco fibers.

### THE INVENTION

The invention relates to an oral nicotine composition, being substantially free from tobacco fibers and the composition comprising, nicotine, such as free base nicotine, nicotine salt and/or nicotine bound to a resin such as polacrilex in an amount of 0.5 mg to 12 mg, such as 1.0 mg to 10 mg, 1.0 mg to 5.0 mg, 1.0 mg to 4.0 mg, 1.0 mg to 3.0 mg or 1.0 mg to 2.0 mg, such as 1.0, 1.5, 2.0, 2.5, 3.0, 3.5, 4.0, 4.5 or 5.0 mg. The amount of nicotine being dependent upon the craving to be relieved as well as the other components present within the composition.

The oral nicotine composition comprises one or more fillers, wherein the filler/fillers is/are present in an amount of at least 30% by weight, such as at least 40, 45, 50, 55, 60, 65, 70, 75% by weight or from 30% by weight to 80% by weight, such as from 30% by weight to 80% by weight 30% by weight to 75% by weight 35% by weight to 65% by weight 40% by weight to 55% by weight, or from 45% by weight to 55% by weight. The filler increases the volume of the composition as well as influences the release of nicotine from the composition. For example, if there is not enough filler all the nicotine will be immediate released and not extended over a time period resulting in an unbalanced product. Examples of fillers include microcrystalline cellulose, cellulose and cellulose derivatives, and mixtures thereof.

In one embodiment the filler is a mixture of microcrystalline cellulose in an amount of 30-75% by weight, such as 35-65% by weight or 40-50% by weight and a natural fiber in an amount of 5-15% by weight, such as microcrystalline cellulose in an amount of 40, 45, 50% by weight and a natural fiber in an amount of 5, 10, 15% by weight.

Examples of natural fibers includes wheat fibers, pea fibers, rice fiber, maize fibers, oat fibers, powdered oat fibers, tomato fibers, barley fibers, rye fibers, sugar beet fibers, buckwheat fibers, potato fibers, cellulose fibers, apple fibers, cocoa fibers, bran fibers, bamboo fibers, and mixtures thereof.

In another embodiment the fibers are at least oat fibers and/or oat powder used alone or in combination with another filler. In one example oat fiber and/or oat powder are mixed with cellulose, such as microcrystalline cellulose (MCC), wherein the filler is a mixture of microcrystalline cellulose in an amount of 30-75% by weight, such as 35-65% by weight or 40-50% by weight and the oat fiber is in an amount of 5-15% by weight, such as microcrystalline cellulose in an amount of 40, 45, 50% by weight and the oat fiber in an amount of 5, 10, 15% by weight. In the case of utilizing oat, the result is a homogenous and stable composition that retain the shape of the product. The oral nicotine composition with oat upon being placed in the mouth, the saliva wet the oat and oat expand the pouch into a pillow structure and the oral nicotine composition give a nice sensory feeling.

Additionally, it is possible to for the personal user to mold the pouch into a suitable personal shape.

Non-limiting examples of usable water-insoluble fibers are the Vitacel® fibers. They are predominantly insoluble fibers that are extracted and isolated from the skeletal substances of renewable raw materials in a complex defined process. Those products have outstanding properties is suitable to be used in combination with nicotine. Those fibers can be obtained from companies such as J. RETTERMAIER & SÖHNE in Germany.

The oral nicotine composition if substantially free from any tobacco fiber and thereby less harmful components are removed that are present in the tobacco fibers, such as cancerogenic agents and some heavy metals naturally present in the tobacco plant or derived from the cultivating of the tobacco plant and accumulated during the growth.

The composition may further comprise one or more humectants, in an amount of from 2 by weight to 25% by weight, such as from 5% by weight to 25% by weight, from 10% by weight to 15% by weight from 7% by weight to 15% by weight, such as 5, 6, 7, 8, 9, 10, 11, 12, 13, 14, 15% or 20% by weight. Examples of humectants are glycerol, alginate, triacetin modified starch, hydroxypropyl cellulose, pectin and xanthan gum or mixtures thereof.

In one embodiment the humectant is glycerol in an amount of 5% by weight to 25% by weight, such as 20% by weight.

The humectant may be present in the oral nicotine composition together with water, wherein water is present in an amount from 5% by weight to 30% by weight, such as from 10% by weight to 30% by weight from 10% by weight to 20% by weight from 7% by weight to 15% by weight, such as 5, 6, 7, 8, 9, 10, 11, 12, 13, 14, 15, 16, 17, 18, 19, 20% by weight. The humectant and water humidify the composition and thus makes it moldable into a composition with relatively high density that can be added into a container such as a pouch during the production of the product and further added into packages. In addition, the presence of those components improves the mouth feeling and eliminates the dryness of the product.

In one embodiment the water is present in an amount of from 5% by weight to 25% by weight and glycerol in an amount of from 5% by weight to 25% by weight and the amount of the glycerol and the water may be in the same amount.

In addition to the humectant, a vegetable oil may be included to increase the humectant activity and improve the oral nicotine composition to a highly improved appearance of such an oral nicotine composition. In one example glycerol and a vegetable oil is mixed and used as a suitable humectant. The vegetable oil may be present in an amount of from 0.1 to 3.0% by weight.

The combination of nicotine, humectant, water and sodium chloride increases the preservation activity and thus in a natural way without using preservatives microbial growth is inhibited.

The oral nicotine composition may as well include one or more sweeteners, thickeners and flavors. Preferably the oral nicotine composition is substantially free from any pH adjusting agent.

Examples of sweeteners include mono- di- tri- and polysaccharides, polyols (sugar alcohols) such as mannitol, sucralose, erythritol, maltitol and xylitol, natural and synthetic sweeteners such as sucrose, glucose, dextrose, maltose, fructose, saccharin, aspartame, acesulfame, neotame, sucralose, monk fruit, stevia, saccharin and cyclamates, and mixtures thereof. The sugar alcohols may be present in an amount of 2% by weight to 15% by weight, such as 5% by weight to 10% by weight 6, 7, 8, 9, 10, 11, 12, 13, 14% by weight alone or a s a mixture of sugar alcohols, wherein the sugar alcohols are mannitol, erythritol or xylitol or mixtures thereof. Other sweeteners, such as sucralose may be present in an amount of 0.1 to 1.0% by weight.

Examples of thickeners include alginate, sodium alginate, pectin or synthetic versions thereof. The thickener may be present in an amount of at most 5% by weight.

The oral nicotine composition further comprises at least one flavor, natural or synthetic. A large numbers of flavors synthetic and non-synthetic are present on the market and available from any is suitable that give the desired flavor of the product. Flavors according to the present invention may include bergamot, eucalyptus, menthol, cinnamon, mint, peppermint, spearmint, apple, cherry, melon, mango, matcha, mocha, ginger-peach, honey-melon, lime, tropical, peach, passion fruit, orange, blood orange, grape fruit, mandarin, tangerine, licorice, ginger, vanilla, wintergreen, lemon, lime, strawberry, blueberry, raspberry, salmonberry, lingonberry, cranberry, blueberry, muscadine berry, sea buckthorn, chocolate, green tea, lavender, viol, cola, acai, coffee, mocha, or mixtures thereof.

In one embodiment the nicotine oral composition comprises silicone dioxide. in an amount of 0.5% by weight to 3% by weight.

In another embodiment the invention relates to an oral nicotine pouch comprising the oral nicotine composition as defined above and a method for preparing such a product.

Such a method comprises the steps of: a) providing the nicotine source, the at least one filler; b) mixing the nicotine source and the filler provided in step a); c) optionally sieving the powder mixture of step b) into a composition; and d) optionally filling the powder composition into a pouch.

### EXAMPLES

The oral nicotine composition is mixed according to general knowledge and packed into pouches.

| Ingredient | Amount % by weight except nicotine (mg) | | | |
|---|---|---|---|---|
| Nicotine | 1 | 1 | 1 | 1 |
| Oat fiber | 10 | 5 | 10 | 5 |
| MCC | 45 | 50 | 45 | 50 |
| Xylitol | 5 | 5 | 2.5 | 2.5 |
| Erytritol | 5 | 5 | 2.5 | 2.5 |
| Water | 15 | 15 | 18 | 18 |
| Glycerol (E422) | 11 | 11 | 13 | 13 |
| NaCl | 5 | 5 | 5 | 5 |
| Peppermint | 3 | 3 | | |
| flavor | | | | |
| Macha | | | 3 | 3 |

1. An oral nicotine composition comprising,
a. nicotine from 0.5 to 12 mg, and
b. a filler, in an amount of 30-75% by weight and selected from the group consisting of microcrystalline cellulose, cellulose and cellulose derivatives, and mixtures thereof and
c. a natural fiber in an amount of 5-15% by weight.
and substantially free from tobacco.

2. The oral nicotine composition according to claim 1, wherein the filler is in an amount of 35-65% by weight.

3. The oral nicotine composition according to claim 1, wherein the filler microcrystalline cellulose.

4. The oral nicotine composition according to claim 1, wherein the natural fiber is oat fiber.

5. The composition according to claim 1, wherein the composition further comprises one or more humectants, in an amount of from 2% by weight to 25% by weight.

6. The composition according to claim 1, wherein the composition further comprises one or more humectants, in an amount of from 10% by weight to 15% by weight.

7. The composition according to claim 1, wherein the composition further comprises water in an amount of from 5% by weight to 30% by weight.

8. The composition according to claim 5, wherein the humectant is selected from the group consisting of glycerol, alginate, triacetin modified starch, hydroxypropyl cellulose, pectin and xanthan gum or mixtures thereof.

9. The composition according to claim 8, wherein the humectant is glycerol.

10. The oral nicotine composition according to claim 1, further comprises one or more sugar alcohols selected from the group consisting of mannitol, sucralose, erythritol and xylitol or mixtures thereof.

11. The oral nicotine composition according to claim 10, wherein the sugar alcohol is in an amount of 2% by weight to 10% by weight.

12. The oral nicotine composition according to claim 1, comprising silicone dioxide.

13. The oral nicotine composition according to claim 12, wherein silicone dioxide is present in an amount of 0.5% by weight to 3% by weight.

14. The oral nicotine composition according to claim 1, wherein the nicotine is selected from the group consisting of free base nicotine, nicotine salt, bound to an ion exchange resin or mixtures thereof.

15. The oral nicotine composition according to claim 14, wherein the nicotine is nicotine salt.

16. The composition according to claim 1, comprising one or more thickeners, sweeteners or flavors.

17. An oral nicotine container comprising the nicotine composition according to claim 1.

18. The oral nicotine container according to claim 17, wherein the container is an oral nicotine pouch.
